(19) United States
(12) Patent Application Publication
(10) Pub. No.: US 2012/0121687 A1
(43) Pub. Date: May 17, 2012

(54) Wound healing material and method for fabricating the same

(75) Inventors: Yung Chang, Chung-Li (TW); Hung-Hsing Hsu, Chung-Li (TW); Kuo-Lun Tung, Chung-Li (TW); Jheng-Fong Jhong, Chung-Li (TW); Ta-Chin Wei, Chung-Li (TW)

(73) Assignee: CHUNG YUAN CHRISTIAN UNIVERSITY, Tao-Yuan (TW)

(21) Appl. No.: 12/945,121

(22) Filed: Nov. 12, 2010

(51) Int. Cl. A61K 9/70; A61P 17/02; B05D 3/04; A61K 31/74

(52) U.S. Cl. 424/447; 424/78.08; 427/2.31

(57) ABSTRACT

The present invention provides a wound healing material and method for fabricating the same. The wound healing material comprises a hydrophobic fluoro-containing membrane having a first surface and a second surface opposing to each other; and at least one biocompatible polymer covalently bonded to at least one part of the first surface of the membrane wherein the membrane is air permeable but liquid impermeable and the water contact angle of the first surface formed with the biocompatible polymer is smaller than or equal to 40 degrees.

### BACKGROUND OF THE INVENTION

1. Field of the Invention

The present invention is generally related to a wound healing material and method for fabricating the same, and more particularly to a wound healing material using an amphiphilic fluoro-containing polymer and method for fabricating the same.

2. Description of the Prior Art

Human skin has a total surface area of about 1.5˜2.0 m² to maintain the temperature and water content of a body and to prevent from bacterial infection and environmental damage. Human skin is composed of three primary layers: epidermis, dermis, and hypodermis. When skin is hurt to create a wound, the wound healing process comprises the following three phases: inflammatory phase, fibroplasias phase, and maturation or remodeling phase. The smoothness of undergoing the three wound healing phases determines whether or not to completely heal the wound, or become incomplete recovery or worse.

The requirements of a wound healing material include, for example, (1) accelerating the wound healing process, (2) being comfortable like real skin to a patient, and (3) producing no scar or as few as possible.

The most commonly used wound healing material is a traditional textile like cotton gauze. Such a material is only for temporarily covering a wound and it is required frequently replacing a new material. Currently commercial products comprise non-occlusive, occlusive or semi-occlusive materials. For example, the non-occlusive material includes hydrogels formed by hydrophilic polymers, such as gelatin, polysaccharides, etc., capable of absorbing exudates. The commercial product of a non-occlusive type comprises for example Vigilon (CR Bard, USA). On the other hand, the occlusive material is usually formed by a thin flexible membrane, such as polyurethane, and an adhesive covering layer to prevent water evaporation from the surface of a wound to keep the wound moist. The commercial product of an occlusive type comprises for example Tegaderm™ (3M, USA). The semi-occlusive material has a higher evaporation rate than the occlusive type and thus the wound treated with the semi-occlusive material is under semi-dried state. The commercial product of a semi-occlusive type comprises for example Omiderm (latro Medical Systems, UK).

Furthermore, the occlusive material having absorption property comprises for example Tegasorb™ (3M, USA, U.S. Pat. No. 4,952,618), Duoderm™ (Convatec, UK), etc., which is a hydrocolloid dressing (HCD) made up of hydrocolioid particles (e.g. consisting of gelatin, pectin, etc.) embedded in a hydrophobic matrix (e.g. a polyisobutylene). These products are usually used to treat chronic ulcerations of the skin. But, according to various reports, it is found that these hydrocolloid dressings may promote wound healing in a short term but their use is often associated with undesirable inflammatory effects or granulation tissue formation.

U.S. Pat. No. 4,952,618 disclosed a hydrocolloid adhesive composition, comprising a rubbery elastomeric base having dispersed therein hydrocolloid particles, at least some of which are polycationic hydrocolloid particles. Chitosan malate or glutamate is used as the polycationic hydrocolloid particle and polyisobutylene is used as the rubbery elastomer. The backing for the hydrocolloid adhesive composition is porous polyethylene or polyurethane having a moisture vapor transmission rate (MVTR) of 500 g/m²/day (measured at 40° C., 80% humidity differential). However, the disadvantages of using polyethylene or polyurethane include being dirty, not antiseptic, and bad permeability.

Therefore, the key to evaluate the wound healing effect of these materials (wound healing materials or dressings) is mainly focused on biocompatibility, promoting epithelization, reducing rejection and skin inflammatory response, having no callus or scar. Currently, no perfect commercially-available product exists. While using these wound healing materials, considering the condition of the wound, medical expense, and product price, the most economical and effective method is usually used to achieve the optimum result. The research of wound healing is expected to have the wound healed and skin regenerated but not patched so as to provide scarless tissues without any callus.

### SUMMARY OF THE INVENTION

In light of the above background, in order to fulfill the industrial requirements, the invention provides a wound healing material and the method for fabricating the same, particularly provides an amphiphilic wound healing material.

One object of the present invention is to provide a wound healing material using an amphiphilic material, the hydrophilic surface of which is close to a wound and the hydrophobic surface of which is in contact with the external environment of the wound, having the characteristics of keeping the wound air-breathable and moist, being water-proof, antiseptic, and anti-coagulation. The antiseptic characteristic can prevent the wound from infection and inflammation during healing process and also keep the wound moist and air-breathable. The wound healing material according to the present invention can accelerate the healing process of a wound and also leave no callus on the new born skin by grafting a specific polymer on a hydrophobic surface of a substrate and adjusting the water contact angle of the surface of the substrate that is close to the wound within a certain range. Thus, the purpose of near perfect wound healing and skin recovery can be achieved. The method of adjusting the water contact angle of the surface can be implemented by grafting biocompatible polymers on the surface. The biocompatible polymers comprise two types of polymers: polymer without any charged moiety and zwitterionic polymer or pseudo-zwitterionic polymer. Preferably, the biocompatible polymers are zwitterionic polymers or pseudo-zwitterionic polymers.

Another object of the present invention is to provide a method for fabricating a wound healing material. The method uses atmospheric plasma treatment to have the biocompatible polymers grafted on the surface of a fluoro-containing membrane so as to fabricate the above mentioned wound healing material of the present invention. The wound healing material has the characteristics of keeping the wound air-breathable and moist, being water-proof, antiseptic, and anti-coagulation.

The fluoro-containing membrane according to the invention is inherently hydrophobic and is an effective insulation material to resist germs, bacteria, micro-particles. The fluoro-containing membrane according to the invention is gas permeable but liquid impermeable and has a moisture vapor transmission rate (MVTR) of at least more than 500 g/m²/day (ASTM E96-80). Therefore, the fluoro-containing membrane grafted with the specific polymers possesses not only the characteristics of the fluoro-containing membrane but also the characteristics of the grafted polymers and thereby has the characteristics of keeping the wound air-breathable and moist, being water-proof, antiseptic, and anti-coagulation while being used as a wound healing material.

Accordingly, the present invention discloses a wound healing material, comprising: a fluoro-containing hydrophobic membrane, having a first surface and a second surface opposing to each other and being gas permeable but liquid impermeable; and at least one biocompatible polymer, covalently bonded to a portion of the first surface of the hydrophobic membrane; wherein the portion of the first surface formed with the biocompatible polymer is hydrophilic. The at least one biocompatible polymer comprises one compound selected from the group consisting of the following or combination thereof: polymer without any charged moiety, zwitterionic polymer and pseudo-zwitterionic polymer.

Furthermore, the present invention discloses a method for fabricating a wound healing material, comprising: providing a fluoro-containing hydrophobic membrane, having a first surface and a second surface opposing to each other and being gas permeable but liquid impermeable; coating a biocompatible polymer precursor solution on the first surface of the hydrophobic membrane; performing a drying process for drying the biocompatible polymer precursor solution on the first surface; and performing atmospheric plasma treatment on the first surface of the hydrophobic membrane to have the biocompatible polymer grafted on first surface so that the first surface of the hydrophobic membrane becomes hydrophilic and the water contact angle of the first surface is less than 40 degrees, preferably, 20˜30 degrees.

In the above method for fabricating a wound healing material, the first surface of the hydrophobic membrane can be processed by surface activation treatment before the step of coating a biocompatible polymer precursor solution. The surface activation treatment can be low pressure plasma treatment or ozone treatment. In one embodiment, the low pressure plasma treatment uses argon plasma.

In conclusion, the wound healing material and the method for fabricating the same according to the invention are disclosed. By using the fluoro-containing membrane grafted with the specific polymers, the present invention has the advantages of keeping the wound air-breathable and moist, being water-proof, antiseptic, and anti-coagulation because of using not only the characteristics of the fluoro-containing membrane but also the characteristics of the grafted polymers. In addition, the method for fabricating a wound healing material according to the invention uses the atmospheric plasma treatment to perform graft polymerization to achieve the purpose of reducing the cost of mass-production. Therefore, the present invention does have the economic advantages for industrial applications.

### BRIEF DESCRIPTION OF THE DRAWINGS

FIG. 1(a) shows a top-view schematic diagram illustrating the structure of the wound healing material according to the invention;

FIG. 1(b) shows a cross-sectional schematic diagram illustrating the structure of the wound healing material according to the invention;

FIG. 2 shows a flow chart of the method for fabricating a wound healing material according to one embodiment of the present invention;

FIGS. 3(a) and (b) show schematic diagrams illustrating the relations of the argon plasma processing time with the grafting density and the water contact angle of the wound healing material when (a) PEGMA and (b) SBMA are grafted to the surface of PTFE membrane, respectively;

FIGS. 4 (a) and (b) show the FTIR spectra of PTFE (raw), PTFE-g-PEGMA, and PTFE-g-SBMA where the peak at 1730 cm⁻¹ represents the absorption peak of C═O group and the peak at 1030 cm⁻¹ represents the absorption peak of —S═O group according to one embodiment of the present invention;

FIG. 5 shows the protein (fibrinogen is used) adsorption test result;

FIG. 6 shows the bacterium (S. epidermidis) adsorption test result of PTFE-g-PEGMA where #1 is PTFE, #2˜#6 are PTFE-g-PEGMA processed by plasma for 5/10/15/60/120 seconds;

FIG. 7 shows the bacterium (E. coli) adsorption test result of PTFE-g-PEGMA where #1 is PTFE, #2˜#6 are PTFE-g-PEGMA processed by plasma for 5/10/15/60/120 seconds;

FIG. 8 shows the bacterium (S. epidermidis) adsorption test result of PTFE-g-SBMA;

FIG. 9 shows the bacterium (E. coli) adsorption test result of PTFE-g-SBMA;

FIG. 10 shows a processing flow diagram of fabricating a wound healing material of PVDF-g-SBMA according to one embodiment of the invention;

FIG. 11 shows the FTIR spectra of PVDF (virgin PVDF), PVDF-OH, and PVDF-g-PSBMA where PVDF-g-PSBMA is processed by plasma for 30/60/90/120 seconds, separately;

FIG. 12 shows the relations of the argon plasma processing time with the grafting density and the water contact angle of the wound healing materials when SBMA is grafted to the surface of PVDF membrane;

FIG. 13 shows the plasma protein adsorption test result of PVDF (virgin PVDF), PVDF-OH, and PVDF-g-PSBMA; and

FIG. 14 shows microscopic images illustrating the animal experimental result by applying the wound healing patches according to the invention on the wounds of a mouse and dissecting the healed mouse tissues after 10 days.

### DESCRIPTION OF THE PREFERRED EMBODIMENTS

What is probed into the invention is a wound healing material. Detail descriptions of the elements and steps will be provided in the following in order to make the invention thoroughly understood. Obviously, the application of the invention is not confined to specific details familiar to those who are skilled in the art. On the other hand, the common elements and steps that are known to everyone are not described in details to avoid unnecessary limits of the invention. Some preferred embodiments of the present invention will now be described in greater detail in the following. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

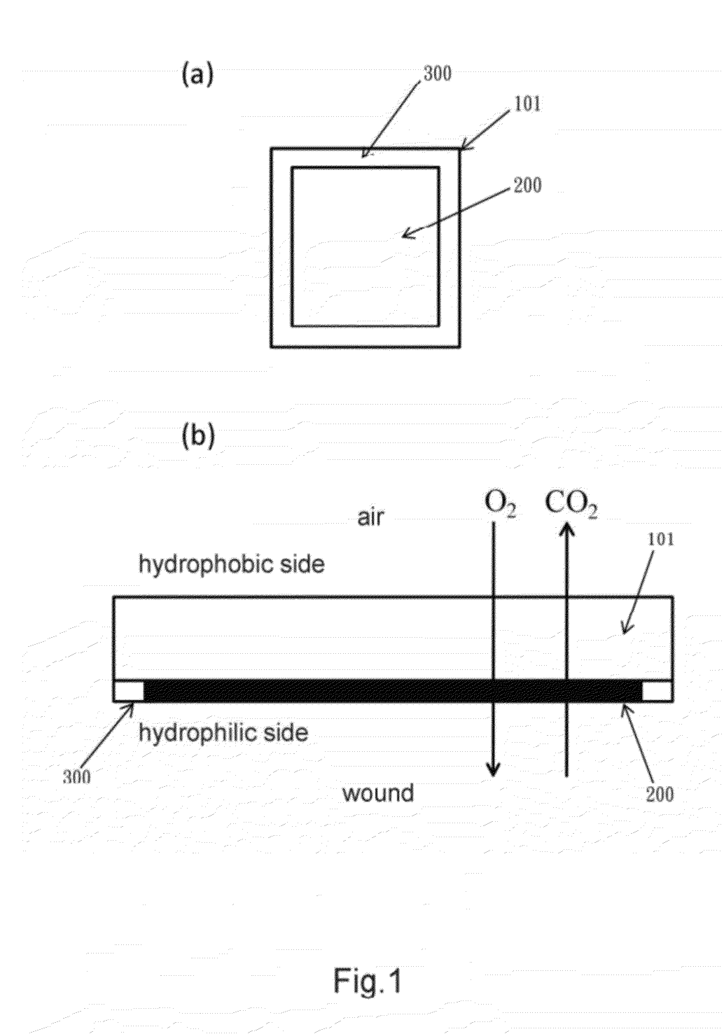

The wound healing material according to the invention is of amphiphilic where the surface attached to the skin is hydrophilic and the other surface (opposing to the surface attached to the skin) exposed to atmosphere is hydrophobic. FIG. 1(a) shows a top-view schematic diagram of the structure of the wound healing material while FIG. 1(b) shows its cross-sectional schematic diagram. The upper part of FIG. 1(b) is the hydrophobic side or the side away from the wound and the lower part of FIG. 1(b) is the hydrophilic side or the side close to the wound. The wound healing material according to the present invention can accelerate the healing process of a wound and also leave no callus on the new born skin by adjusting the water contact angle of the surface of the substrate that is close to the wound within a certain range. The method to adjust the water contact angle uses specific biocompatible polymers to graft on the surface close to the wound. The specific biocompatible polymers comprise two types of polymers, that is, polymer without any charged moiety and zwitterionic polymer or pseudo-zwitterionic polymer. Preferably, the biocompatible polymers are zwitterionic polymers or pseudo-zwitterionic polymers.

A first embodiment of the invention discloses a wound healing material, comprises a fluoro-containing hydrophobic membrane and at least one biocompatible polymer. The fluoro-containing hydrophobic membrane has a first surface and a second surface opposing to each other and is gas permeable but liquid impermeable. The at least one biocompatible polymer, covalently bonded to a portion of the first surface of the hydrophobic membrane. The portion of the first surface formed with the biocompatible polymer is hydrophilic. The water contact angle of the portion of the first surface formed with the biocompatible polymer is less than or equal to 40 degrees, preferably 10˜40 degrees, more preferably 20˜30 degrees. Particularly, when the water contact angle is within 20˜30 degrees, the effect of no callus is more prominent. The second surface is hydrophobic and the water contact angle is more than or equal to 100 degrees.

In one embodiment, the at least one biocompatible polymer comprises one compound selected from the group consisting of the following or combination thereof: polymer without any charged moiety, zwitterionic polymer and pseudo-zwitterionic polymer. For example, the at least one biocompatible polymer is polyethylene glycol methacrylate (PEGMA) or polysulfobetaine methacrylate (PSBMA). The example of the pseudo-zwitterionic polymer comprises a polymer formed by polymerization of positively charged moieties and negatively charged moieties with a molar ratio of 1 to 1.

The positively charged moieties can be, for example,

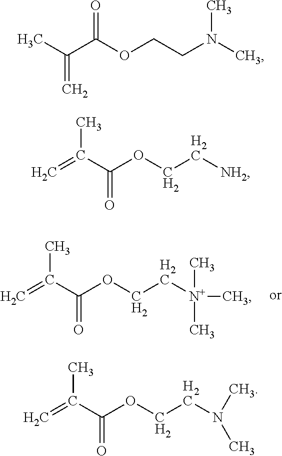

The negatively charged moieties can be, for example,

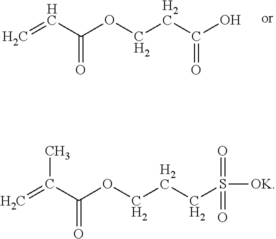

In one embodiment, the at least one biocompatible polymer is graft polymerized to the first surface. When the first surface is formed with PEGMA, if the grafting density of PEGMA is between 0.03 mg/cm² and 0.2 mg/cm², the wound healing effect as a wound healing material is optimum. When the first surface is formed with SBMA, if the grafting density of SBMA is between 0.05 mg/cm² and 0.2 mg/cm², the wound healing effect as a wound healing material is optimum.

When the above wound healing material is tested for the bacterium adsorption test, for example, using E. coli or S. epidermidis, the number of bacteria adsorbed on the first surface is less than or equal to 1%. Moreover, when the above wound healing material is tested for the protein adsorption test, the number of proteins adsorbed on the first surface is less than or equal to 1%.

The above fluoro-containing hydrophobic membrane is polytetrafluoroethylene (PTFE) or polyvinylidene fluoride (PVDF) and has a moisture vapor transmission rate (MVTR) of at least more than 500 g/m²/day. The thickness of the fluoro-containing hydrophobic membrane is for example 10˜500 pan, preferably 30˜300 μm, and more preferably 50˜150 μm.

The method for forming the biocompatible polymer on the first surface of the hydrophobic membrane should be specially designed to bond these two materials because usually the hydrophilic material and the hydrophobic material are difficult to be chemically bonded with each other. Thus, according to the invention, the surface of the hydrophobic membrane is firstly activated and then the plasma or ozone treatment is used to perform plasma induced polymerization to thereby achieve the effect of chemically bonding the hydrophilic polymer on the surface of the hydrophobic membrane.

A second embodiment of the invention discloses a method for fabricating a wound healing material, comprising the following steps. At first, a fluoro-containing hydrophobic membrane is provided where the hydrophobic membrane has a first surface and a second surface opposing to each other and is gas permeable but liquid impermeable. A biocompatible polymer precursor solution is coated on the first surface of the hydrophobic membrane. A drying process is performed to dry the biocompatible polymer precursor solution on the first surface. Finally, atmospheric plasma treatment on the first surface of the hydrophobic membrane is performed to have the biocompatible polymer grafted on first surface so that the first surface of the hydrophobic membrane becomes hydrophilic and the water contact angle of the first surface is less than 40 degrees, preferably 10˜40 degrees, more preferably 20˜30 degrees. In addition, the second surface is hydrophobic and the water contact angle is more than or equal to 100 degrees.

The above fluoro-containing hydrophobic membrane is polytetrafluoroethylene (PTFE) or polyvinylidene fluoride (PVDF) and has a moisture vapor transmission rate (MVTR) of at least more than 500 g/m²/day. The thickness of the fluoro-containing hydrophobic membrane is for example 10˜500 μm, preferably 30˜300 μm, and more preferably 50˜150

In one embodiment, the at least one biocompatible polymer comprises one compound selected from the group consisting of the following or combination thereof: polymer without any charged moiety, zwitterionic polymer and pseudo-zwitterionic polymer. For example, the at least one biocompatible polymer is polyethylene glycol methacrylate (PEGMA) or polysulfobetaine methacrylate (PSBMA). The example of the pseudo-zwitterionic polymer comprises a polymer formed by polymerization of positively charged moieties and negatively charged moieties with a molar ratio of 1 to 1. The examples of the positively charged moieties and the negatively charged moieties can be the same as those shown in the first embodiment.

The water contact angle of the first surface is decreased with the increase of the processing time of the atmospheric plasma treatment. The water contact angle of the first surface is less than 40 degrees, preferably 10˜40 degrees, more preferably 20˜30 degrees. The above atmospheric plasma treatment uses argon plasma to process the first surface.

A third embodiment of the invention discloses a method for fabricating a wound healing material, comprising the following steps. The difference between the methods of the third embodiment and the second embodiment is that in the third embodiment a surface activation treatment on the first surface of the hydrophobic membrane is performed before coating the biocompatible polymer precursor solution. In one embodiment, the surface activation treatment is low pressure plasma treatment or ozone treatment. In one embodiment, the above atmospheric plasma treatment uses argon plasma to process and the processing time is more than or equal to 60 seconds. Besides, the above low pressure plasma treatment uses argon plasma to process and the processing time is more than or equal to 60 seconds.

### Example 1

### Fabricating PTFE-g-PEGMA/SBMA/TM/SA Wound Healing Materials

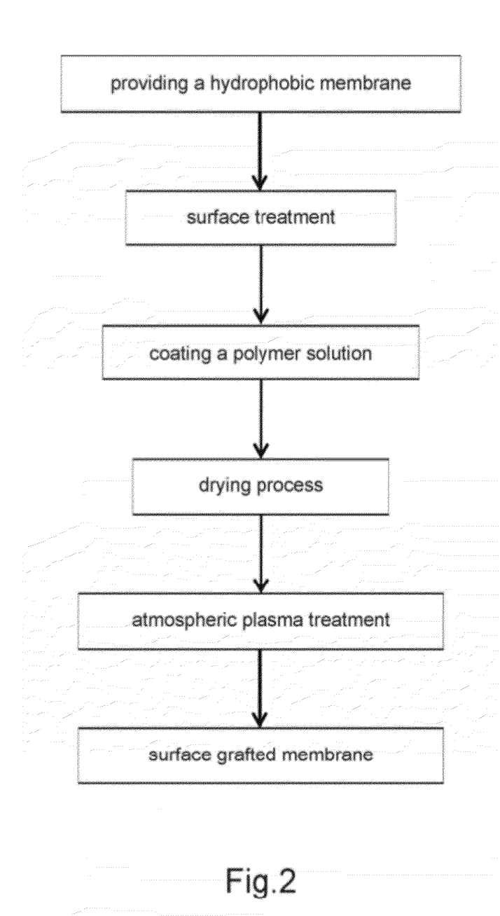

FIG. 2 shows a flow chart of the method for fabricating a wound healing material. PTFE membranes are used as hydrophobic membranes to fabricate the PTFE membrane grafted with (a) PEGMA (polyethylene glycol methacrylate), (b) SBMA (polysulfobetaine methacrylate or PSBMA), (c) TM ([2-(Methacryloyloxy)ethyl]-trimethylammonium chloride), and SA (3-Sulfopropyl methacrylate potassium salt) on one surface where (c) and (d) are used as the control groups. The first surface of each PTFE membrane is an inactive hydrophobic surface. After the PTFE membranes are then placed in a vacuum chamber and the first surface of each membrane is processed by argon plasma (power 150 W for 60 seconds), the PTFE membranes are exposed in air at 40° C. for 10 min. 30 wt % of PEGMA (or SBMA) solution is coated on the first surface and dried. Then, the first surface coated with the polymer (PEGMA or SBMA) solution is processed by argon plasma under atmospheric pressure for the processing time of 5, 15, 30, 60, or 120 seconds to perform graft polymerization on the first surface to separately covalently bond PEGMA/SBMA on the first surface of each PTFE membrane.

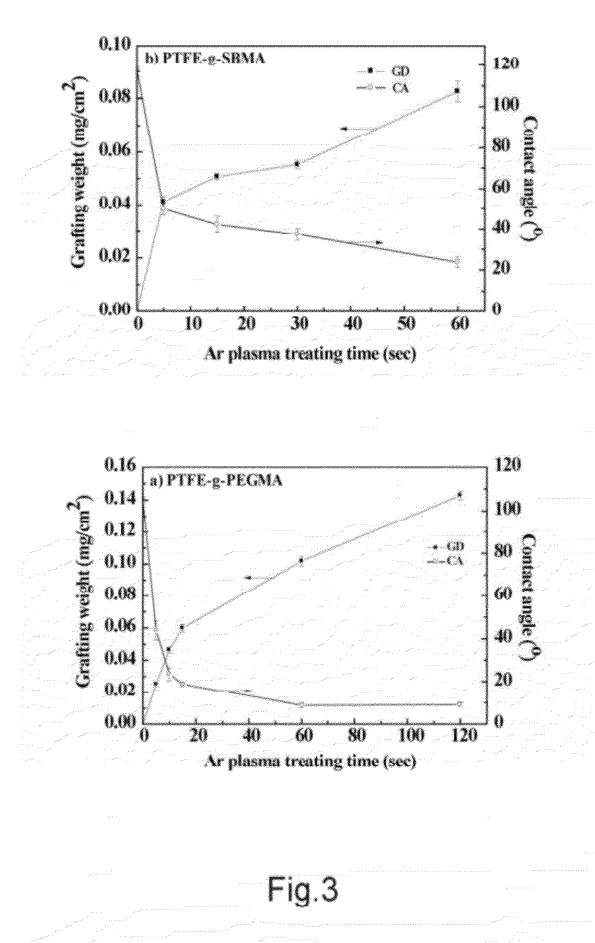

FIGS. 3(a) and (b) show schematic diagrams illustrating the relations of the argon plasma processing time with the grafting density and the water contact angle of the wound healing material when (a) PEGMA and (b) SBMA are grafted to the surface of PTFE membrane, respectively. From the figure, the grafting density of the wound healing material is increased and the water contact angle is decreased with the increase of the processing time of the argon plasma treatment.

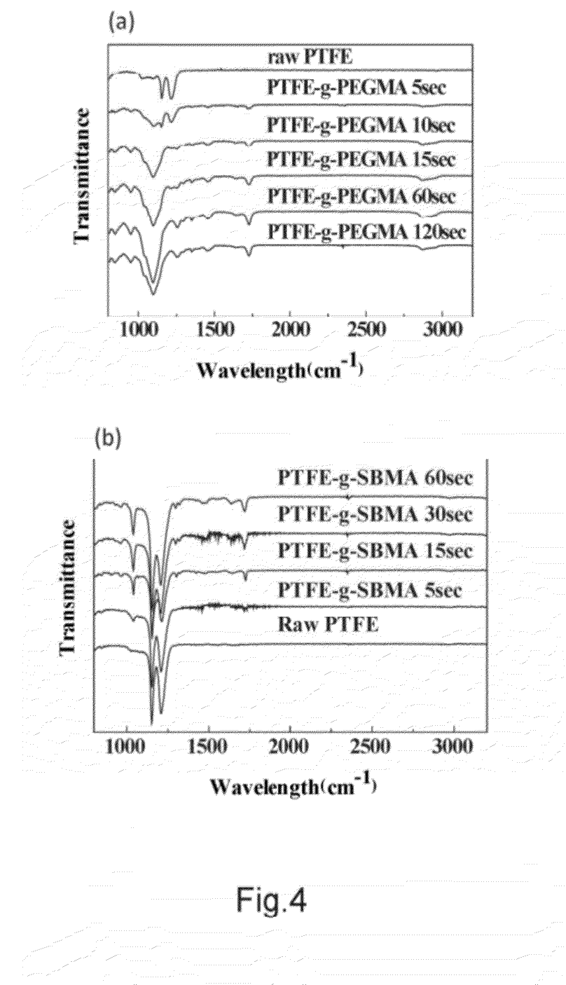

FIGS. 4 (a) and (b) show the FTIR spectra of PTFE (raw), PTFE-g-PEGMA, and PTFE-g-SBMA where the peak at 1730 cm⁻¹ represents the absorption peak of C═O group and the peak at 1030 cm⁻¹ represents the absorption peak of —S═O group.

The following will perform the characteristic tests of the wound healing material (PTFE-gPEGMA/SBMA).

Protein Adsorption Test

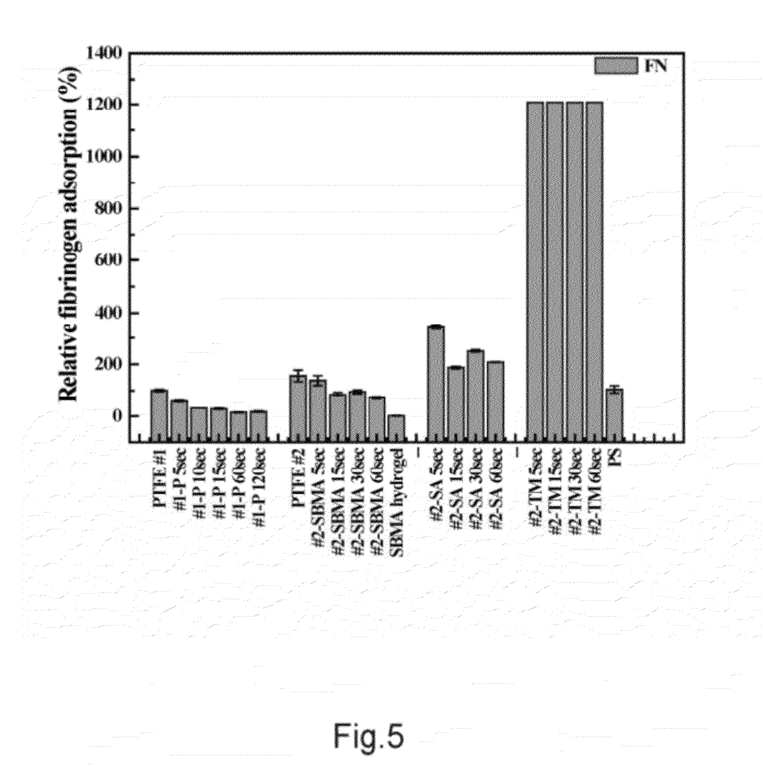

Fibrinogen is used as an example of proteins to test the adsorption characteristic of PTFE-g-PEGMA/SMMA/TM/SA. FIG. 5 shows the protein (fibrinogen is used) adsorption test result. On the horizontal axis, #1-P 5/10/15/60/120 sec indicates that the PEGMA is grafted on the PTFE surface and processed for 5/10/15/60/120 sec; #2-SBMA 5/10/15/60 sec indicates that the SBMA is grafted on the PTFE surface and processed for 5/10/15/60 sec; #2-SA 5/10/15/60 sec indicates that the SA is grafted on the PTFE surface and processed for 5/10/15/60 sec; #2-TM 5/10/15/60 sec indicates that the TM is grafted on the PTFE surface and processed for 5/10/15/60 sec; and PS indicates polysulfone. From FIG. 5, the fibrinogen adsorption percentage of the surface grafted with PEGAM, SBMA, and SA is less than that of TM.

Bacterium Adsorption Test

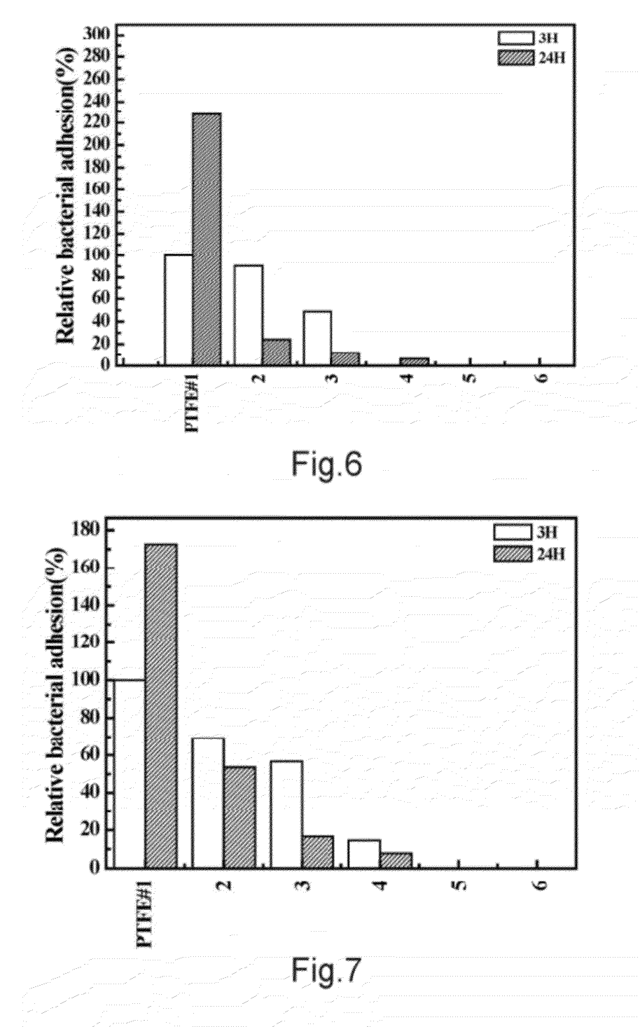

S. epidermidis and E. coli are used as examples of bacteria to test the adsorption characteristic of PTFE-g-PEGMA/SMMA/TM/SA. FIG. 6 shows the bacterium (S. epidermidis) adsorption test result of PTFE-g-PEGMA where #1 is PTFE, #2˜#6 are PTFE-g-PEGMA processed by plasma for 5/10/15/60/120 seconds. FIG. 7 shows the bacterium (E. coli) adsorption test result of PTFE-g-PEGMA where #1 is PTFE, #2˜#6 are PTFE-g-PEGMA processed by plasma for 5/10/15/60/120 seconds. From FIGS. 6 and 7, compared with the pristine PTFE, the bacterium adsorption of the processed PTFE (PTFE grafted with PEGMA) and is remarkably decreased.

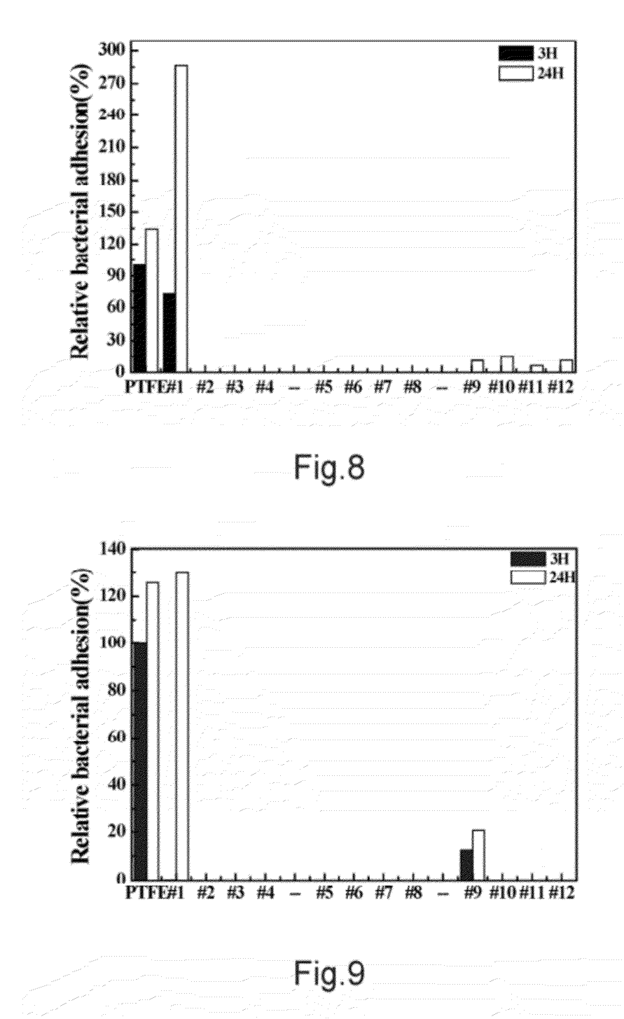

On the other hand, FIG. 8 shows the bacterium (S. epidermidis) adsorption test result of PTFE-g-SBMA and FIG. 9 shows the bacterium (E. coli) adsorption test result of PTFE-g-SBMA. The samples are in the order of the unprocessed PTFE, PTFE-g-SMBA 5/15/30/60 sec (#1˜#4), PTFE-g-SA 5/15/30/60 sec (#5˜#8), and PTFE-g-TM 5/15/30/60 sec (#9˜#12). From FIGS. 8 and 9, compared with the pristine PTFE, the bacterium adsorption of the processed PTFE (PTFE grafted with SBMA) and is remarkably decreased.

Although the above example 1 uses PTFE membranes as the fluoro-containing hydrophobic membranes, PVDF membranes can be used as well.

### Example 2

### Method for Fabricating a Wound Healing Material

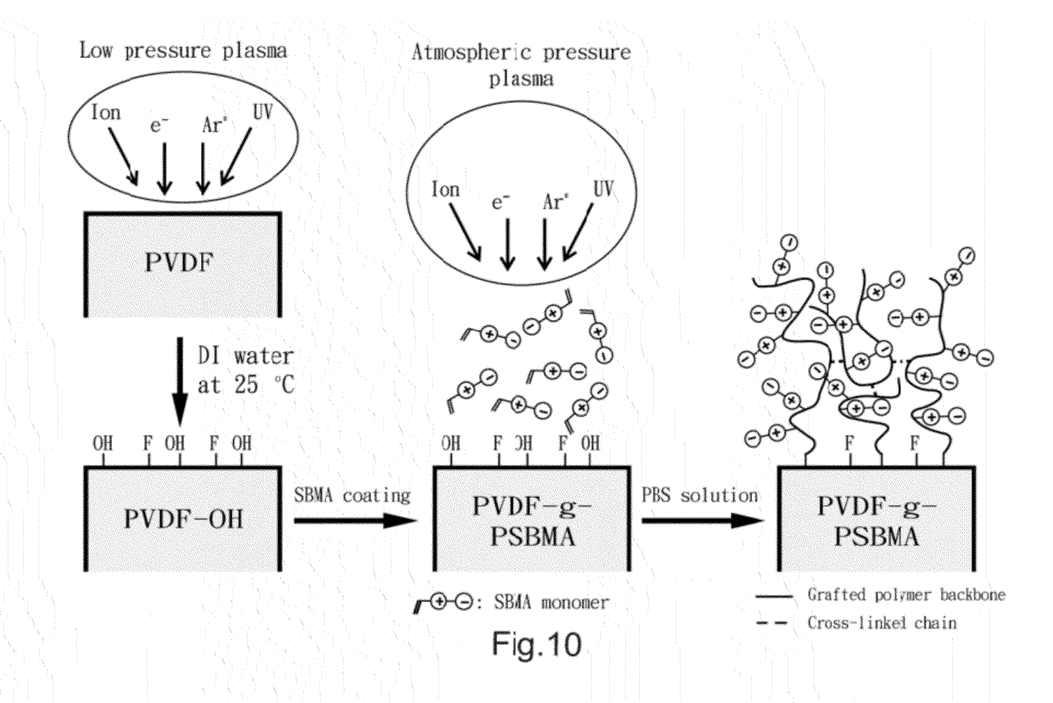

The following will describe an example of using PVDF membranes as the fluoro-containing hydrophobic membranes to fabricate the PVDF-g-SBMA wound healing material. FIG. 10 shows a processing flow diagram of fabricating a wound healing material of PVDF-k-SBMA.

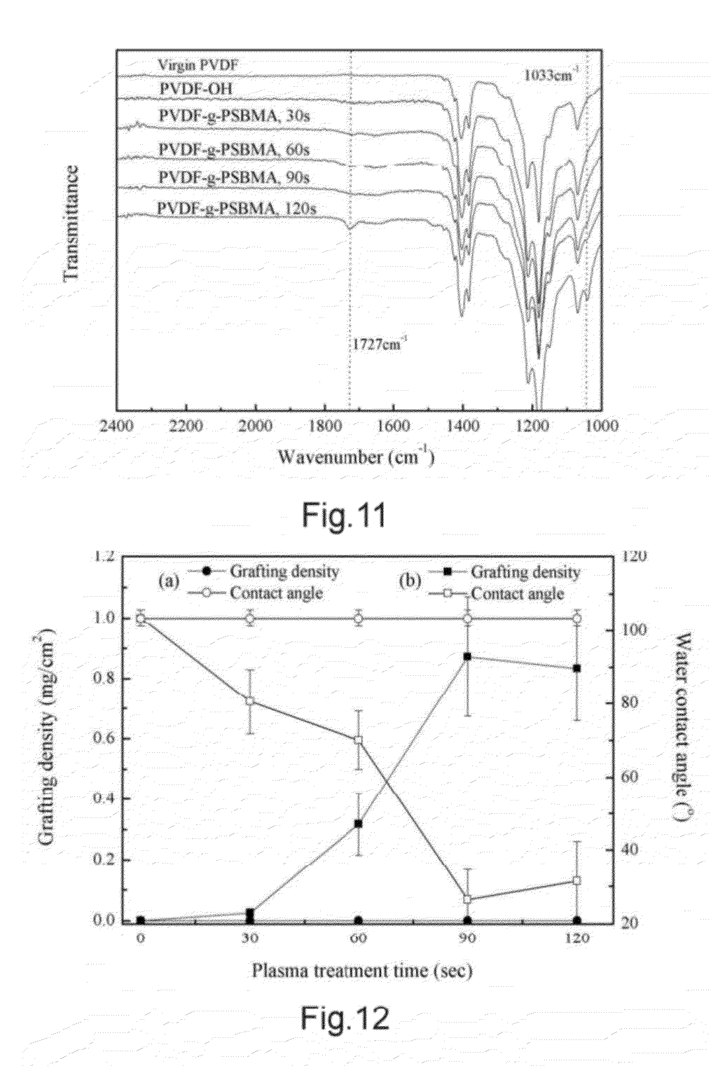

FIG. 11 shows the FTIR spectra of PVDF (virgin PVDF), PVDF-OH, and PVDF-g-PSBMA where PVDF-g-PSBMA is processed by plasma for 30/60/90/120 seconds, separately.

FIG. 12 shows the relations of the argon plasma processing time with the grafting density and the water contact angle of the wound healing materials when SBMA is grafted to the surface of PVDF membrane.

Protein Adsorption Test

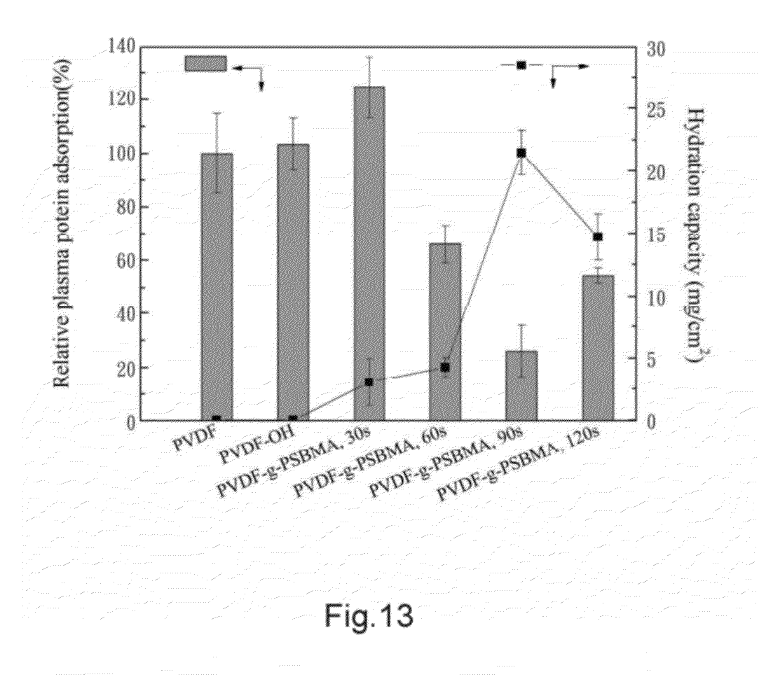

FIG. 13 shows the plasma protein adsorption test result of PVDF (virgin PVDF), PVDF-OH, and PVDF-g-PSBMA illustrating the relation between the plasma protein adsorption percentage and the processing time of the plasma treatment.

### Example 3

### Fabricating Wound Healing Patches

The wound healing material prepared by the method in example 1 is used where the PTFE membrane grafted with PEGAM or SBMA (PTFE-g-PEGMA or PTFE-g-SBMA) is formed. As shown in FIG. 1, PEGMA or SBMA are grafted on the center portion of the PTFE surface and a pressure sensitive adhesive layer, such as polyvinyl ether adhesive and copolymer acrylate, is formed on the peripheral portion of the surface. In FIG. 1, the shape of the area grafted with PEGMA or SBMA is a rectangle and is only an example. Other shapes, such as circular, oval, or any polygonal shape, can be used. The shape of the membrane can be any other shape, such as circular, oval, or any polygonal shape, besides the rectangular shape shown in FIG. 1.

Animal Experiment of the Wound Healing Patches

The above prepared wound healing patches according to the invention are used as the test samples to cover the wounds of a mouse (wound size 1.5×1.5 cm²) and then adhesive tapes being water-proof and air-breathable are used to secure the patches on the wounds without falling off. After 10 days, the wound sections are observed. Besides, a cotton gauze, a commercial product (3M made), a pristine PTFE (labeled as PTFE), super-hydrophobic treated PTFE (labeled as CF4) are used as the control groups.

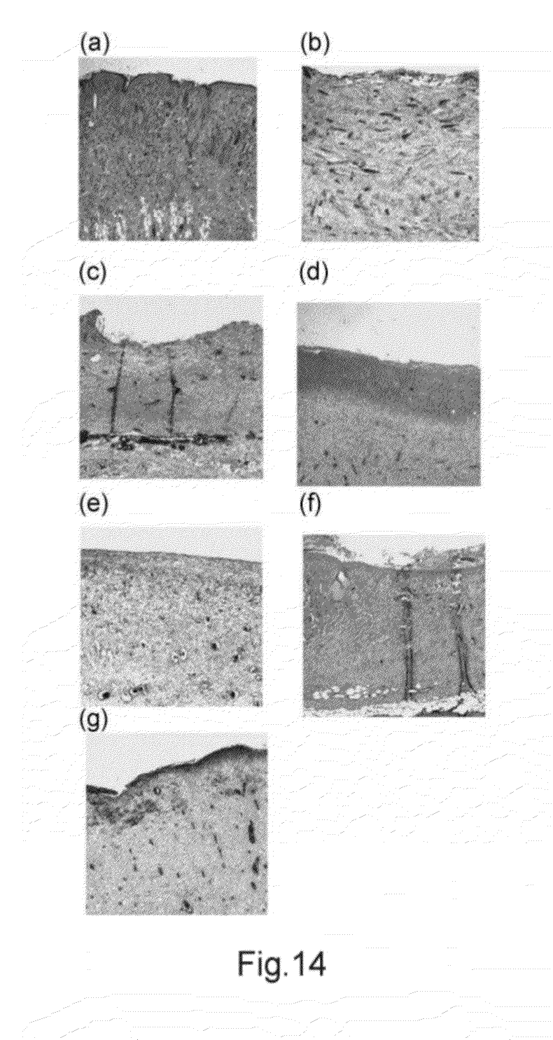

FIG. 14 shows microscopic images illustrating the animal experimental result by applying the wound healing patches according to the invention on the wounds of a mouse and dissecting the healed mouse tissues after 10 days where (a): normal skin; (b): cotton gauze; (c): commercial product; (d) pristine PTFE (labeled as PTFE); (e): super-hydrophobic treated PTFE (labeled as CF4); (f): PTGE-g-SBMA (labeled as SBMA); and (g) PTFE-g-PEGMA (labeled as PEGMA, processed 10 sec).

From FIG. 14(b), the cotton gauze is used to cover the wound and bleeding is still observed. It is found that loose tissues are formed and the growth of new blood capillaries is not remarkable. From FIG. 14(c), the wound section after using the commercial product shows grown dermis tissues, no callus formed, and no epidermis tissue but hyperplasia of blood capillaries is apparent. Besides, there is a regularly-arranged fibrous structure formed on the wound. From FIG. 14(d), a plurality of immunocytes aggregate on the upper part of the wound and a thin layer of calluses is grown on the topmost portion. From the 40× section image in the figure, hyperplasia of blood capillaries can be seen in the deeper tissues. From FIG. 14(e), the section of the wound after using the super-hydrophobic treated PTFE membrane shows that a thin layer of epidermis tissues starts to undergo differentiation and a plurality of blood capillaries undergo differentiation in the wound tissues.

From FIG. 14(f), the section image of the wound after using PTFE-g-SBMA according to the invention shows that epidermis tissues undergo differentiation and is formed and the hypodermis is almost close to a state of recovery.

From FIG. 14(g), the section image of the wound after using PTFE-g-PEGMA processed 10 sec according to the invention shows that calluses are formed on the top of the wound but there no serious immunocyte aggregation. Besides, hyperplasia of epidermis and blood capillaries is observed.

The following Table 1 shows the evaluation result of the above animal experiments. The method of evaluation is based on the hyperplasia of calluses, blood capillaries, immunocytes, and epidermis where the number of “X”s indicates the quantity of hyperplasia and more “X”s represent that the hyperplasia is more apparent. Since the recovery of a wound is better when grown skin tissues are closer to the original skin tissues, the hyperplasia of blood capillaries and epidermis is preferred and the hyperplasia of calluses and immunocytes is disfavored.

TABLE 1
the evaluation result of the animal experiments

| | | blood | | |
|---|---|---|---|---|
| sample | callus | capillary | immunocyte | epidermis |
| Cotton gauze | | X | XXX | |
| Commercial | | XXX | X | |
| product | | | | |
| PTFE | X | XX | XXX | |
| CF4 | | XXX | X | XX |
| SBMA | | XXX | | XXX |
| PEGMA-10s | XX | XXX | X | X |

From Table 1, the PTFE-g-SBMA patch according to the invention has an excellent wound healing result that has no callus and immunocyte grown and epidermis is grown. Compared to the commercial product, the grown skin tissues are almost near recovery when the PTFE-g-SBMA patch according to the invention is used and the recovery rate is also faster.

In conclusion, the wound healing material and the method for fabricating the same according to the invention are disclosed. By using the fluoro-containing membrane grafted with the specific polymers, the present invention has the advantages of keeping the wound air-breathable and moist, being water-proof, antiseptic, and anti-coagulation because of using not only the characteristics of the fluoro-containing membrane but also the characteristics of the grafted polymers. In addition, the method for fabricating a wound healing material according to the invention uses the atmospheric plasma treatment to perform graft polymerization to achieve the purpose of reducing the cost of mass-production.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

What is claimed is:

1. A wound healing material, comprising:
a fluoro-containing hydrophobic membrane, having a first surface and a second surface opposing to each other and being gas permeable but liquid impermeable; and
at least one biocompatible polymer, covalently bonded to a portion of the first surface of the hydrophobic membrane;
wherein the portion of the first surface formed with the biocompatible polymer is hydrophilic.

2. The material according to claim 1, wherein the at least one polymer comprises one compound selected from the group consisting of the following or combination thereof: polymer without any charged moiety, zwitterionic polymer and pseudo-zwitterionic polymer.

3. The material according to claim 2, wherein the at least one polymer comprises polyethylene glycol methacrylate (PEGMA).

4. The material according to claim 2, wherein the at least one polymer comprises polysulfobetaine methacrylate (PSBMA).

5. The material according to claim 2, wherein the pseudo-zwitterionic polymer is formed by polymerization of positively charged moieties and negatively charged moieties with a molar ratio of 1 to 1.

6. The material according to claim 5, wherein the positively charged moieties are selected from the group consisting of the following:

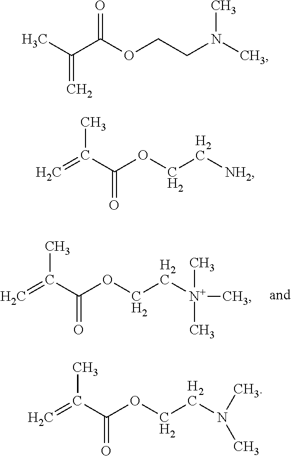

7. The apparatus according to claim 5, wherein the negatively charged moieties are selected from the group consisting of the following:

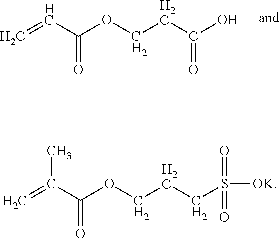

8. The material according to claim 1, wherein the water contact angle of the portion of the first surface formed with the biocompatible polymer is smaller than 40 degrees.

9. The material according to claim 1, wherein the water contact angle of the portion of the first surface formed with the biocompatible polymer is within 20˜30 degrees.

10. The material according to claim 1, wherein in the portion of the first surface formed with the biocompatible polymer the grafting density of the polymer is more than or equal to 0.03 mg/cm² and less than or equal to 0.2 mg/cm².

11. The material according to claim 1, wherein the number of bacteria adsorbed on the portion of the first surface formed with the biocompatible polymer while the wound healing material is tested in a bacterium adsorption test is less than or equal to 1%.

12. The material according to claim 1, wherein the number of proteins adsorbed on the portion of the first surface formed with the biocompatible polymer while the wound healing material is tested in a protein adsorption test is less than or equal to 1%.

13. The material according to claim 1, wherein the fluoro-containing hydrophobic membrane is polytetrafluoroethylene (PTFE) or polyvinylidene fluoride (PVDF).

14. A method for fabricating a wound healing material, comprising:
providing a fluoro-containing hydrophobic membrane, having a first surface and a second surface opposing to each other and being gas permeable but liquid impermeable;
coating a biocompatible polymer precursor solution on the first surface of the hydrophobic membrane;
performing a drying process for drying the biocompatible polymer precursor solution on the first surface; and
performing atmospheric plasma treatment on the first surface of the hydrophobic membrane to have the biocompatible polymer grafted on first surface so that the first surface of the hydrophobic membrane becomes hydrophilic and the water contact angle of the first surface is less than 40 degrees.

15. The method according to claim 14, wherein the fluoro-containing hydrophobic membrane is polytetrafluoroethylene (PTFE) or polyvinylidene fluoride (PVDF).

16. The method according to claim 14, wherein the at least one polymer comprises one compound selected from the group consisting of the following or combination thereof: polymer without any charged moiety, zwitterionic polymer and pseudo-zwitterionic polymer.

17. The method according to claim 14, wherein the biocompatible polymer precursor solution comprises polyethylene glycol methacrylate (PEGMA) or polysulfobetaine methacrylate (PSBMA).

18. The method according to claim 14, wherein the processing time of the atmospheric plasma treatment is adjusted to have the water contact angle of the first surface be within 20˜30 degrees.

19. The method according to claim 14, wherein the atmospheric plasma treatment uses argon plasma.

20. A method for fabricating a wound healing material, comprising:
providing a hydrophobic membrane, having a first surface and a second surface opposing to each other and being gas permeable but liquid impermeable;
performing a surface activation treatment on the first surface;
coating a biocompatible polymer precursor solution on the first surface of the hydrophobic membrane; and
performing atmospheric plasma treatment on the first surface of the hydrophobic membrane to have the biocompatible polymer grafted on first surface so that the first surface of the hydrophobic membrane becomes hydrophilic and the water contact angle of the first surface is less than 40 degrees.

21. The method according to claim 20, wherein the surface activation treatment is low pressure plasma treatment or ozone treatment.

22. The method according to claim 20, wherein the fluoro-containing hydrophobic membrane is polytetrafluoroethylene (PTFE) or polyvinylidene fluoride (PVDF).

23. The method according to claim 20, wherein the processing time of the atmospheric plasma treatment is adjusted to have the water contact angle of the first surface be within 20˜30 degrees.

24. The method according to claim 20, wherein the at least one polymer comprises one compound selected from the group consisting of the following or combination thereof: polymer without any charged moiety, zwitterionic polymer and pseudo-zwitterionic polymer.

25. The method according to claim 20, wherein the biocompatible polymer precursor solution comprises polyethylene glycol methacrylate (PEGMA) or polysulfobetaine methacrylate (PSBMA).

26. The method according to claim 20, wherein the atmospheric plasma treatment uses argon plasma and the processing time of the atmospheric plasma treatment is more than or equal to 60 seconds.

27. The method according to claim 21, wherein the low pressure plasma treatment uses argon plasma and the processing time of the low pressure plasma treatment is more than or equal to 60 seconds.
